(19) United States
(12) Patent Application Publication
(10) Pub. No.: US 2025/0269123 A1
(43) Pub. Date: Aug. 28, 2025

(54) REAL-TIME BREATH ADAPTIVE NEBULIZER APPARATUS AND METHODS FOR PULMONARY THERAPEUTIC AGENT DELIVERY

(71) Applicant: BOARD OF REGENTS, THE UNIVERSITY OF TEXAS SYSTEM, Austin, TX (US)

(72) Inventors: Hugh SMYTH, Austin, TX (US); Matthew HERPIN, Austin, TX (US)

(21) Appl. No.: 18/858,186

(22) PCT Filed: Apr. 19, 2023
(86) PCT No.: PCT/US2023/065933

§ 371 Date: Oct. 18, 2024

Related U.S. Application Data
(60) Provisional application No. 63/332,392, filed on Apr. 19, 2022.

(51) Int. Cl. A61M 15/00; A61M 16/00

(52) U.S. Cl. CPC A61M 15/0085; A61M 15/0086; A61M 2016/0039; A61M 2205/3334; A61M 2205/50

(57) ABSTRACT

Apparatus and methods to apparatus and methods for optimizing pulmonary delivery of therapeutic agent. Exemplary embodiments include an inlet for airflow a reservoir containing a therapeutic agent and an airflow sensor configured to detect the airflow through the apparatus. In addition, exemplary embodiments include an aerosol generator configured to generate an aerosol of the therapeutic agent, an outlet configured to deliver the aerosol to a user, and a control module configured to receive an input signal from the airflow sensor and provide an output signal to the aerosol generator.

### CROSS-REFERENCE TO RELATED APPLICATIONS

This application claims priority to U.S. Provisional Patent Application Ser. No. 63/332,392 filed Apr. 19, 2022, the entire contents of which are incorporated by reference herein.

### BACKGROUND INFORMATION

Embodiments of the present invention relate to apparatus and methods for apparatus and methods for optimal pulmonary therapeutic agent delivery.

Inhalation of aerosols for delivery to the respiratory tract is well known to be influenced by particle or droplet size of the aerosol, as well as the inhalation velocity. More specifically, particles and droplets of different sizes will deposit within different regions of the lungs (i.e upper and lower airways).

The location where these particles are deposited is also affected by the inhalation maneuver performed by the user. The inconsistency or variability in this maneuver is a leading cause for delivered dose variability. The inhalation maneuver can vary greatly between different users based on factors such as age, physical condition, etc. There can also be variations in the inhalation maneuver performed by the same user under different conditions. Existing apparatus that utilize pre-set parameters for aerosol generation do not accommodate for variations in inhalation airflow, and therefore may not create an appropriate droplet size for optimal therapeutic benefit to the user.

Accordingly, a need exists to provide for consistent pulmonary therapeutic agent delivery in a manner that accounts for variations in the inhalation maneuver performed by the user.

### SUMMARY

Exemplary embodiments of the present disclosure include features that enable the pulmonary therapeutic agent aerosol that is being emitted to be altered in real-time in response to changes in the inhalation profile. Such features allow the inhalation device to be utilized in a broad patient population and be of particular benefit for those patients with the inability to inhale with normal pulmonary function (e.g. patients suffering from lung disease).

Exemplary embodiments include an apparatus for optimal pulmonary therapeutic agent delivery, where the apparatus comprises: an inlet for airflow; a reservoir containing a therapeutic agent; an airflow sensor configured to detect the airflow through the apparatus; an aerosol generator configured to generate an aerosol of the therapeutic agent; an outlet configured to deliver the aerosol to a user; and a control module configured to receive an input signal from the airflow sensor and provide an output signal to the aerosol generator, where: the apparatus is configured to produce a first droplet size of the aerosol at a first flow rate of the airflow; the apparatus is configured to produce a second droplet size of the aerosol at a second flow rate of the airflow; the first droplet size is larger than the second droplet size; and the first flow rate is lower than the second flow rate.

In certain embodiments the aerosol generator comprises a mesh screen, and in particular embodiments the mesh screen is configured to generate aerosol droplets between 1 μm and 10 μm when vibrated. In certain embodiments, the mesh screen comprises a first region comprising a first pore size, and the mesh screen comprises a second region comprising a second pore size. In particular embodiments, the first region and the second region are each configured to generate aerosol droplets with a diameter between 1 μm and 10 μm when vibrated. In specific embodiments, the first region and the second region can be independently activated to generate aerosol droplets.

In some embodiments the aerosol generator comprises a plurality of mesh screens. In specific embodiments the plurality of mesh screens comprises: a first mesh screen comprising a first mesh pore size; a second mesh screen comprising a second mesh pore size; and the second mesh pore size is different than the first mesh pore size. In certain embodiments the first mesh screen and the second mesh screen are each configured to generate aerosol droplets with a diameter between 1 μm and 10 μm when vibrated.

In particular embodiments the first mesh screen is a first distance from the outlet; the second mesh screen is a second distance from the outlet; and the first distance is greater than the second distance. In some embodiments the plurality of mesh screens comprises a third mesh screen. In specific embodiments the first mesh screen, the second mesh screen and the third mesh screen are each configured to generate aerosol droplets between 1 μm and 10 μm when vibrated. In certain embodiments the first mesh screen is configured to generate aerosol droplets with a diameter of approximately 1 μm when vibrated, the second mesh screen is configured to generate aerosol droplets with a diameter of approximately 5 μm when vibrated and the third mesh screen is configured to generate aerosol droplets with a diameter of approximately 10 μm when vibrated.

In particular embodiments the third mesh screen comprises a third mesh pore size, and the third mesh screen size is different than the first mesh pore size and the second mesh pore size. In some embodiments the aerosol comprises droplets, and wherein during use the control module is configured to adjust the output signal to control a median diameter of the droplets in the aerosol.

Exemplary embodiments comprise a method of controlling droplet size in a pulmonary therapeutic aerosol, where the method comprises: measuring an inlet airflow in a nebulizer apparatus, providing an input signal to the control module, wherein the input signal is dependent on the inlet airflow; providing an output signal from the control module to the aerosol generator to generate an aerosol of droplets of the therapeutic agent; and adjusting the output signal to control droplet size of the therapeutic agent. In specific embodiments the nebulizer apparatus comprises: an inlet, a control module, a therapeutic agent, an aerosol generator, and an outlet.

In certain embodiments the droplets of the therapeutic agent have a diameter of between approximately 1 μm and 10 μm. In particular embodiments the aerosol generator comprises a mesh screen, and generating the aerosol of droplets of the therapeutic agent comprises vibrating the mesh screen. In some embodiments adjusting the output signal comprises altering a frequency of the output signal. In specific embodiments adjusting the output signal comprises altering a voltage of the output signal. In certain embodiments adjusting the output signal comprises altering a waveform of the output signal. In particular embodiments the mesh screen is a first mesh screen in a plurality of mesh screens of the aerosol generator. In some embodiments the plurality of mesh screens comprises: the first mesh screen comprising a first mesh pore size; a second mesh screen comprising a second mesh pore size; and the second mesh pore size is different than the first mesh pore size.

In particular embodiments of the method, the first mesh screen and the second mesh screen are each configured to generate aerosol droplets with a diameter between 1 μm and 10 μm when vibrated. In some embodiments the first mesh screen is a first distance from the outlet; the second mesh screen is a second distance from the outlet; and the first distance is greater than the second distance. In specific embodiments the plurality of mesh screens comprises a third mesh screen. In certain embodiments the first mesh screen, the second mesh screen and the third mesh screen are each configured to generate aerosol droplets between 1 μm and 10 μm when vibrated. In particular embodiments the first mesh screen is configured to generate aerosol droplets with a diameter of approximately 1 μm when vibrated, the second mesh screen is configured to generate aerosol droplets with a diameter of approximately 5 μm when vibrated and the third mesh screen is configured to generate aerosol droplets with a diameter of approximately 10 μm when vibrated. In some embodiments the third mesh screen comprises a third mesh pore size, and the third mesh screen size is different than the first mesh pore size and the second mesh pore size. In specific embodiments the output signal is configured to vibrate one or more of the plurality of mesh screens.

In the present disclosure, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The terms “approximately, “about” or “substantially” mean, in general, the stated value plus or minus 10%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 shows a schematic view of an apparatus according to an exemplary embodiment of the present disclosure.

FIG. 2 shows an overview of aspects of an exemplary method according to the present disclosure.

FIG. 3 shows a partial schematic view of an apparatus according to the present disclosure under different operating conditions and the associated aerosol distribution for each operating condition.

FIG. 4 shows a partial schematic view of an apparatus according to the present disclosure under different operating conditions and the associated aerosol distribution for each operating condition.

FIG. 5 shows a partial schematic view of an apparatus according to the present disclosure under different operating conditions and the associated aerosol distribution for each operating condition.

### DETAILED DESCRIPTION OF EXEMPLARY EMBODIMENTS

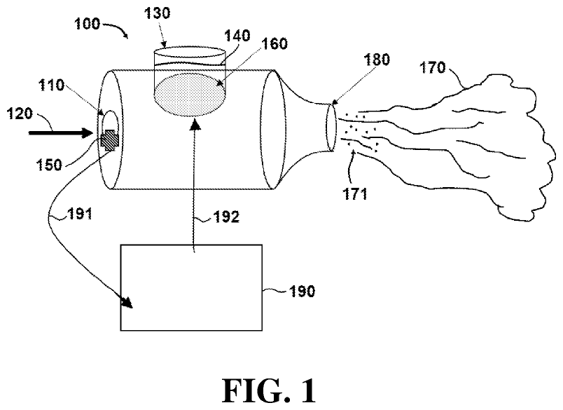

Embodiments of the present disclosure include apparatus and methods for optimal pulmonary therapeutic agent delivery. Referring initially to FIG. 1, a schematic view of an apparatus 100 for optimal pulmonary delivery of a therapeutic agent 140 is shown. In this embodiment, apparatus 100 comprises an inlet 110 for airflow 120, as well as a reservoir 130 containing therapeutic agent 140. In addition, the illustrated embodiment comprises an airflow sensor 150 configured to detect airflow 120 through the apparatus 100, and an aerosol generator 160 configured to generate an aerosol 170 of droplets 171 of therapeutic agent 140. In exemplary embodiments, airflow sensor 150 may be any form of sensor configured to detect changes in airflow, including for example, pressure, temperature or optical sensors. In particular embodiments, airflow sensor 150 may be a TSI™ Alnor® 41403 Mass Flowmeter for Gases

Apparatus 100 also comprises an outlet 180 configured to deliver aerosol 170 to a user (not shown). In this embodiment, apparatus 100 further comprises a control module 190 configured to receive an input signal 191 from airflow sensor 150 and provide an output signal 192 to aerosol generator 160. In exemplary embodiments, the user may form a seal around outlet 180 and inhale to draw airflow 120 from inlet 110 through apparatus 100 to the user. In certain embodiments, control module 190 can be configured to automatically generate output signal 192 upon detection of input signal 191. In other embodiments, a user may manually activate apparatus 100 (e.g. by pressing a button or switch) to allow control module 190 to transmit output signal 192 to aerosol generator 160. In particular embodiments control module 190 may comprise an Arduino® microcontroller or Rasberry® Pi. In certain embodiments, apparatus 100 may utilize control and command software, including for example, National Instruments® Labview or Python based platforms, including MicroPython and CircuitPython.

During operation, control module 190 can receive input signal 191 from airflow sensor 150 and adjust parameters of output signal 192 in response to input signal 191. In certain embodiments, aerosol generator 160 may comprise one or more mesh screens that are vibrated to create an aerosol comprising therapeutic agent 140. In such embodiments, control module 190 can modulate parameters of output signal 192 sent to aerosol generator 160 so that the vibrating mesh screen or screens generate different size droplets 171 in aerosol 170. Accordingly, control module 190 can control the operation of aerosol generator 160 and the delivery of aerosol 170 to optimize therapeutic benefits provided to the user by aerosol 170.

In particular embodiments, control module 190 may modulate the frequency, voltage and waveform of output signal 192 provided to aerosol generator 160. Accordingly, control module can be configured to adjust output signal 192 in response to input signal 191 to control the size (e.g. the diameter or volume) of the droplets 171 in aerosol 170 during operation. In exemplary embodiments, control module 190 can adjust output signal 192 in real-time to respond to changes from input signal 191 from airflow sensor 150. As such, apparatus 100 can adjust the size of droplets 171 to accommodate variations in airflow 120 created by different users. In specific embodiments, apparatus 100 is configured to produce larger droplet sizes at lower flow rates and smaller droplets at higher flow rates. For example, when sensor 150 detects an increase in the flow rate of airflow 120, control module 190 can transmit an output signal 192 to aerosol generator 160 which results in a decrease in the size of droplets 171. Conversely, if sensor 150 detects a decrease in flow rate of airflow 120, control module 190 can transmit an output signal 192 to aerosol generator 160 which results in an increase in the size of droplets 171.

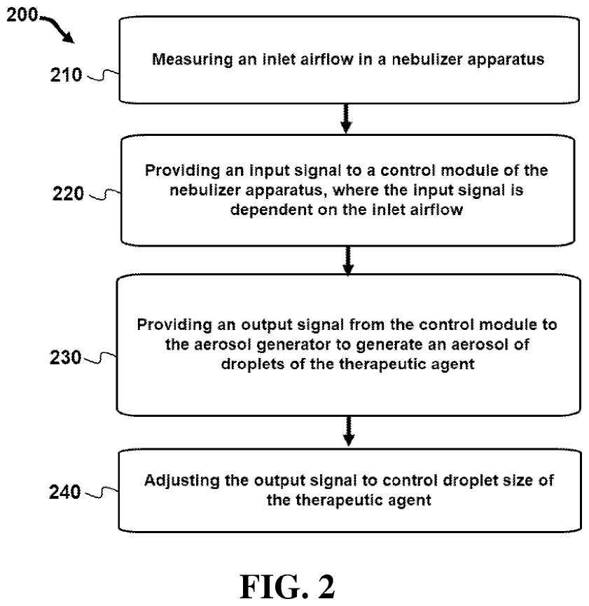

An overview of exemplary method 200 according to the present disclosure is shown in FIG. 2. In the embodiment shown, method 200 comprises an aspect 210 of measuring an inlet airflow in a nebulizer apparatus. Method 200 also comprises an aspect 220 of providing an input signal to a control module of the nebulizer apparatus, where the input signal is dependent on the inlet airflow. Method 200 further comprises an aspect 230 of providing an output signal from the control module to the aerosol generator to generate an aerosol of droplets of the therapeutic agent. In addition, method 200 comprises an aspect 240 of adjusting the output signal to control droplet size of the therapeutic agent.

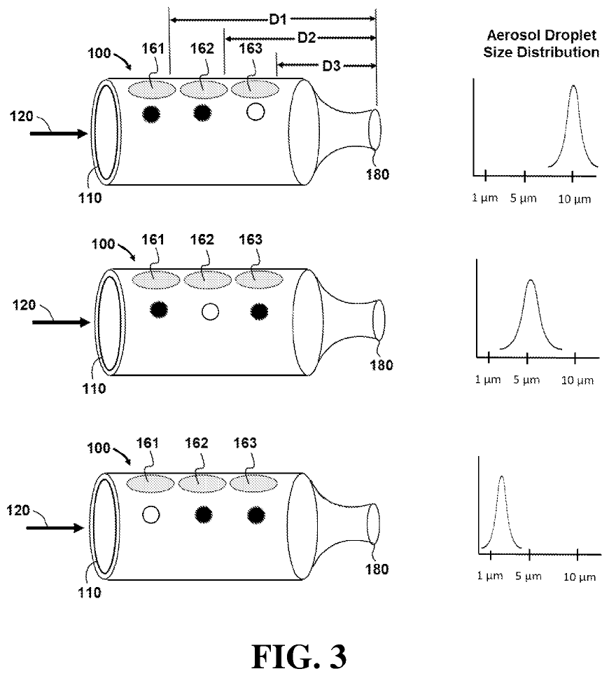

In particular embodiments, aerosol generator 160 may comprise a plurality of mesh screens with different pore sizes and/or different locations to provide different size droplets in aerosol 170. For example, referring now to FIGS. 3-5, embodiments of apparatus 100 are schematically illustrated comprising mesh screens 161, 162 and 163. For purposes of clarity, not all features of apparatus 100 are shown in FIGS. 3-5, including for example, control module 190 and one or more reservoirs 130 containing therapeutic agent 140. As used herein, droplet size may be measured by any relevant quantifiable parameter, including for example, droplet volume or droplet diameter. In specific embodiments apparatus 100 is configured to produce an aerosol 170 with d50 equal to 1, 5 and/or 10 μm and d90 equal to 2, 7 and/or 12 μm (where d50 represents a droplet size distribution such that 50 percent of the droplets are smaller than the specified size, and where d90 represents a droplet size distribution such that 90 percent of the droplets are smaller than the specified size).

In certain embodiments, aerosol generator 160 may comprise different components for different droplet sizes. For example, for small droplet sizes, aerosol generator 160 may comprise an Aerogen® Solo or PDAP vibrating mesh nebulizer, for medium droplet sizes, aerosol generator 160 may comprise a MicroAir® Omron NE-U100, Aerogen® Pro Lab vibrating mesh nebulizer or Tekceleo® Nebulizer H-T45-M04 (4 micron kit), and for large droplet sizes, aerosol generator 160 may comprise a Nephron® EZ-Breathe Atomizer or Tekceleo® Nebulizer H-T45-M12 (12 micron kit).

In the embodiment shown in FIG. 3, each mesh screen 161, 162 and 163 have an equivalent pore size, but are located different distances from outlet 180. Specifically, mesh screen 161 is located a first distance D1 from outlet 180, mesh screen 162 is located a second distance D2 from outlet 180 and third mesh screen 163 is located a third distance D3 from outlet 180. In the embodiments shown in FIGS. 3-5, the circular indicators below mesh screens 161-163 indicate whether or not the mesh screen is activated (e.g. being vibrated via an output signal from control module 190). A white circle indicates a mesh screen that is activated, while a black circle indicates a mesh screen that is not activated. In FIG. 3, the top example shows mesh screen 163 activated, the center example shows mesh screen 162 activated, and the bottom example shows mesh screen 161 activated. The embodiment shown in FIG. 3 also comprises an inlet 110 that is open-ended (e.g. such that the inlet does not provide a significant restriction to air flow 120 entering apparatus 100 during use and provides for dilution and/or evaporation of aerosol droplets as the droplets move from inlet 110 towards outlet 180).

As shown in the graphs for each scenario in FIG. 3, the distance from the activated mesh screen 161, 162 or 163 affects the aerosol droplet size distribution. In particular, when the activated mesh screen is closer to outlet 180, the aerosol droplet size is increased. Conversely, when the activated mesh screen is farther from outlet 180, the aerosol droplet size is decreased. Increasing the distance between the active mesh screen and outlet 180 allows for the aerosol droplets to evaporate into smaller fractions. Accordingly, the aerosol droplet size can be controlled by activating mesh screens that are different distances from outlet 180. In the representative example shown in FIG. 3, the median droplet diameter can be controlled between 10 μm, 5 μm, and 1 μm by activating mesh screen 163, 162 or 161, respectively. While mesh screens are indicated in the embodiment shown in FIGS. 3-5, it is understood that other configurations of aerosol generators are also within the scope of the present disclosure. It is also understood that the droplet sizes shown in FIGS. 3-5 are for comparison purposes, and that other embodiments of the present disclosure may generate different size droplets.

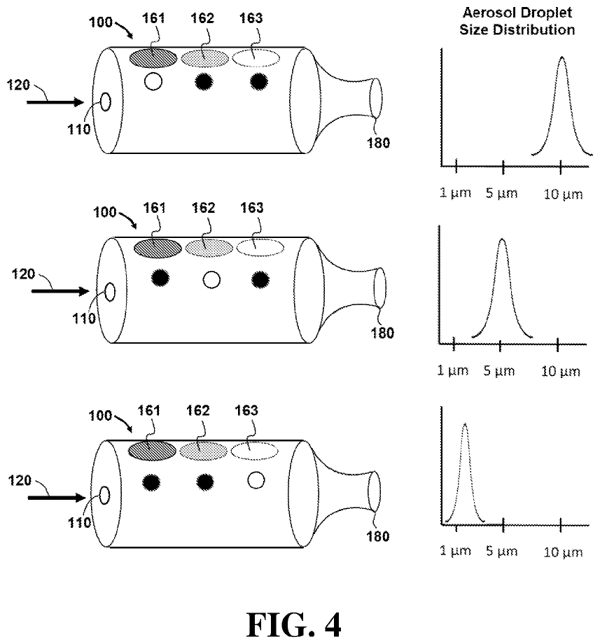

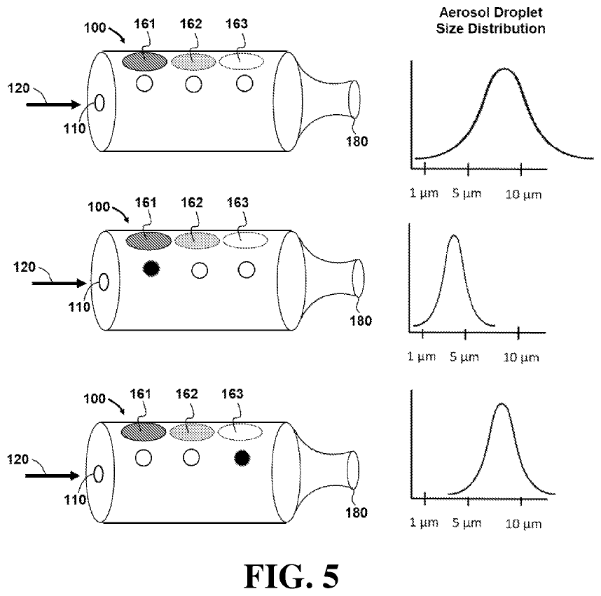

In certain embodiments, other parameters of apparatus 100 can also be used to control the droplet size for aerosol 170. For example, in the embodiment shown in FIG. 4, mesh screens 161, 162 and 163 are not only located different distances from outlet 180, but also comprise different mesh pore sizes. In the embodiment shown in FIGS. 4 and 5, mesh screen 161 has a larger pore size than mesh screen 162, which in turn has a larger pore size than mesh screen 163. In addition, the embodiments shown in FIGS. 4 and 5 comprise an inlet 110 that is reduced in size, creating an enclosed chamber in apparatus 100 that does not provide for dilutive evaporation of aerosol droplets. Accordingly, the distance between the activated mesh screen and outlet 180 does not have a significant impact on the droplet size in this embodiment.

In the top example shown in FIG. 4, only screen 161 (e.g. the screen with the largest port diameter and farthest from outlet 180) is activated, providing for a median droplet size of approximately 10 μm. In the middle example shown in FIG. 4, only screen 162 is activated, providing for a median droplet size of approximately 5 μm. The bottom example shown in FIG. 4 illustrates activation of screen 163 only, generating a median droplet size of slightly more than 1 μm.

FIG. 5 illustrates examples with multiple screens of different mesh sizes and different distances from outlet 180 being activated at the same time. Similar to the embodiment shown in FIG. 4, in this embodiment air inlet 110 is reduced in size, creating an enclosed chamber in apparatus 100 that does not provide for dilutive evaporation of aerosol droplets as the droplets move toward outlet 180.

In the top example, all three mesh screens 161, 162 and 163 are activated. This configuration generates a broader droplet size distribution with a median size between 5 and 10 μm. In the middle example, only screens 162 and 163 are activated, resulting in a droplet size distribution with a median size between 1 and 5 μm. In the bottom example shown in FIG. 5, screens 161 and 162 are activated resulting in a droplet size distribution with a median size between 5 and 10 μm, but with a narrower droplet size distribution than the top example in which all mesh screens are activated. Accordingly, the exemplary embodiments disclosed herein can accommodate for variations in inhalation airflow, and create an appropriate droplet size for optimal therapeutic benefit to the user.

In certain embodiments, patient inhalation data from sensor 150 is fed into control module 190 and presets for flow rates and thresholds are set to trigger individual aerosol generators to be turned on and off according to an inhalation profile. While exemplary embodiments are disclosed herein, input/output signals to and from control module 190 could be done any number of ways using various software and hardware configurations.

In particular embodiments, the aerosol generator mesh screens can be pre-fabricated to generated aerosols with particular characteristics, and an aerosol generator can be tuned for its output by altering formulation characteristics. For example, the reservoirs of the aerosol generator(s) can each filled with a different therapeutic agent formulation to create a different droplet size distribution. In addition, sensors tracking the duration that each aerosol generator/formulation (AG/F) combination is activated can allow for the all the AG/Fs to be turned off when the desired dose of therapeutic agent has been administered.

In addition to particle evaporation and mesh pore size, the physicochemical characteristics of the formulation (e.g. viscosity, surface tension, and/or suspension primary particle/droplet size) can be used for tuning the aerosol distribution. Other aerosol distribution tuning parameters can include the aerosol chamber geometry and/or fluid dynamics, including for example, chamber volume, inlet size for tuning dilutive and evaporative air intake, and sheath air flow inlet to prevent aerosol deposition on walls of chamber.

Accordingly, the exemplary embodiments disclosed herein can accommodate for variations in inhalation airflow, and create an appropriate droplet size and dosage profile for optimal therapeutic benefit to the user.

### REFERENCES

The contents of the following references are incorporated by reference herein:
Pilcer G, Amighi K. Formulation strategy and use of excipients in pulmonary drug delivery. International journal of pharmaceutics. 2010 Jun. 15; 392(1-2):1-9.
Newhouse M T, Hirst P H, Duddu S P, Walter Y H, Tarara T E, Clark A R, Weers J G. Inhalation of a dry powder tobramycin PulmoSphere formulation in healthy volunteers. Chest. 2003 Jul. 1; 124(1):360-6.
Zhang G, David A, Wiedmann T S. Performance of the vibrating membrane aerosol generation device: Aeroneb Micropump Nebulizer™. Journal of aerosol medicine. 2007 Dec. 1; 20(4):408-16.
O'Riordan T G. Formulations and nebulizer performance. Respiratory care. 2002 Nov. 1; 47(11):1305-2.

1. An apparatus for optimal pulmonary therapeutic agent delivery, the apparatus comprising:
an inlet for airflow;
a reservoir containing a therapeutic agent;
an airflow sensor configured to detect the airflow through the apparatus;
an aerosol generator configured to generate an aerosol of the therapeutic agent;
an outlet configured to deliver the aerosol to a user; and
a control module configured to receive an input signal from the airflow sensor and provide an output signal to the aerosol generator, wherein:
the apparatus is configured to produce a first droplet size of the aerosol at a first flow rate of the airflow;
the apparatus is configured to produce a second droplet size of the aerosol at a second flow rate of the airflow;
the first droplet size is larger than the second droplet size; and
the first flow rate is lower than the second flow rate.

2. The apparatus of claim 1 wherein the aerosol generator comprises a mesh screen.

3. The apparatus of claim 1 or claim 2 wherein the mesh screen is configured to generate aerosol droplets between 1 μm and 10 μm when vibrated.

4. The apparatus of claim 2 or claim 3 wherein:
the mesh screen comprises a first region comprising a first pore size; and
the mesh screen comprises a second region comprising a second pore size.

5. The apparatus of claim 4 wherein:
the first region and the second region are each configured to generate aerosol droplets with a diameter between 1 μm and 10 μm when vibrated.

6. The apparatus of claim 4 or claim 5 wherein the first region and the second region can be independently activated to generate aerosol droplets.

7. The apparatus of any one of claims 1-6 wherein the aerosol generator comprises a plurality of mesh screens.

8. The apparatus of claim 7 wherein the plurality of mesh screens comprises:
a first mesh screen comprising a first mesh pore size;
a second mesh screen comprising a second mesh pore size; and
the second mesh pore size is different than the first mesh pore size.

9. The apparatus of claim 8 wherein the first mesh screen and the second mesh screen are each configured to generate aerosol droplets with a diameter between 1 μm and 10 μm when vibrated.

10. The apparatus of claim 8 or claim 9 wherein:
the first mesh screen is a first distance from the outlet;
the second mesh screen is a second distance from the outlet; and
the first distance is greater than the second distance.

11. The apparatus of any one of claims 8-10 wherein the plurality of mesh screens comprises a third mesh screen.

12. The apparatus of claim 11 wherein the first mesh screen, the second mesh screen and the third mesh screen are each configured to generate aerosol droplets between 1 μm and 10 μm when vibrated.

13. The apparatus of claim 11 wherein the first mesh screen is configured to generate aerosol droplets with a diameter of approximately 1 μm when vibrated, the second mesh screen is configured to generate aerosol droplets with a diameter of approximately 5 μm when vibrated and the third mesh screen is configured to generate aerosol droplets with a diameter of approximately 10 μm when vibrated.

14. The apparatus of claim 11 wherein:
the third mesh screen comprises a third mesh pore size; and
the third mesh screen size is different than the first mesh pore size and the second mesh pore size.

15. The apparatus of any one of claims 1-14 wherein the aerosol comprises droplets, and wherein during use the control module is configured to adjust the output signal to control a median diameter of the droplets in the aerosol.

16. A method of controlling droplet size in a pulmonary therapeutic aerosol, the method comprising:
measuring an inlet airflow in a nebulizer apparatus, wherein the nebulizer apparatus comprises:
an inlet;
a control module;
a therapeutic agent;
an aerosol generator; and
an outlet;
providing an input signal to the control module, wherein the input signal is dependent on the inlet airflow;
providing an output signal from the control module to the aerosol generator to generate an aerosol of droplets of the therapeutic agent; and
adjusting the output signal to control droplet size of the therapeutic agent.

17. The method of claim 16 wherein the droplets of the therapeutic agent have a diameter of between approximately 1 μm and 10 μm.

18. The method of claim 16 wherein:
the aerosol generator comprises a mesh screen; and
generating the aerosol of droplets of the therapeutic agent comprises vibrating the mesh screen.

19. The method of any one of claims 16-18 wherein adjusting the output signal comprises altering a frequency of the output signal.

20. The method of any one of claims 16-18 wherein adjusting the output signal comprises altering a voltage of the output signal.

21. The method of any one of claims 16-18 wherein adjusting the output signal comprises altering a waveform of the output signal.

22. The method of any one of claims 16-21 wherein the mesh screen is a first mesh screen in a plurality of mesh screens of the aerosol generator.

23. The method of claim 22 wherein the plurality of mesh screens comprises:
the first mesh screen comprising a first mesh pore size;
a second mesh screen comprising a second mesh pore size; and
the second mesh pore size is different than the first mesh pore size.

24. The method of claim 23 wherein the first mesh screen and the second mesh screen are each configured to generate aerosol droplets with a diameter between 1 μm and 10 μm when vibrated.

25. The method of any one of claims 22-23 wherein:
the first mesh screen is a first distance from the outlet;
the second mesh screen is a second distance from the outlet; and
the first distance is greater than the second distance.

26. The method of claim 22 wherein the plurality of mesh screens comprises a third mesh screen.

27. The method of claim 26 wherein the first mesh screen, the second mesh screen and the third mesh screen are each configured to generate aerosol droplets between 1 μm and 10 μm when vibrated.

28. The method of claim 26 wherein the first mesh screen is configured to generate aerosol droplets with a diameter of approximately 1 μm when vibrated, the second mesh screen is configured to generate aerosol droplets with a diameter of approximately 5 μm when vibrated and the third mesh screen is configured to generate aerosol droplets with a diameter of approximately 10 μm when vibrated.

29. The method of claim 26 wherein:
the third mesh screen comprises a third mesh pore size; and
the third mesh screen size is different than the first mesh pore size and the second mesh pore size.

30. The method of any one of claims 22-29 wherein the output signal is configured to vibrate one or more of the plurality of mesh screens.
